(19) United States
(12) Patent Application Publication
(10) Pub. No.: US 2005/0013884 A1
(43) Pub. Date: Jan. 20, 2005

(54) Compositions and methods for treating heart disease

(76) Inventor: M. Scott Rennels, Glasford, IL (US)

Correspondence Address: BANNER & WITCOFF, LTD., TEN SOUTH WACKER DRIVE, SUITE 3000, CHICAGO, IL, 60606, US

(21) Appl. No.: 10/789,830

(22) Filed: Feb. 27, 2004

Related U.S. Application Data
(60) Provisional application No. 60/487,872, filed on Jul. 16, 2003.

(51) Int. Cl. A61K 35/78; A61K 31/685; A61K 31/525; A61K 31/51; A61K 31/355

(52) U.S. Cl. 424/757; 514/12; 514/78; 514/52; 514/251; 514/276; 514/350; 514/355; 514/458; 514/474; 514/725

(57) ABSTRACT

Nutritional compositions and methods of using the compositions for reducing the likelihood of the occurrence of and/or ameliorating the symptoms of heart disease. The compositions include proteins, phospholipids, vitamins and minerals and other nutritional ingredients. The compositions can be administered on a regular basis to promote good health and treat heart disease, including atherosclerosis.

### CROSS REFERENCE TO RELATED APPLICATION

This application is based on, and claims the benefit of co-pending U.S. Provisional Patent Application Ser. No. 60/487,872, filed Jul. 16, 2003, the disclosures of which is incorporated herein by reference.

### BACKGROUND OF THE INVENTION

There are over 90 million people who suffer from some form of heart disease. According to the American Medical Society and the American Heart Association, heart disease is the number one cause of death in the world. Although heredity and gender factor into whether an individual will suffer from heart disease, lifestyle also impacts the rate and occurrence of heart disease. Poor diet, smoking and lack of exercise, for example, each provide a significant risk with respect to heart disease.

There are approximately 70,000 miles of vessels in the vascular network of the human body. These vessels include arteries, which carry blood away from the heart and veins, and also return blood to the heart. Capillaries make up the majority of the body's vascular network. Most are finer than a strand of human hair. Their walls are usually only a single layer of cells, so that nutrients and other products picked up through the walls of the small intestine can pass through the blood stream and flow to all parts of the body for distribution. Capillaries are innumerable and bring blood into contact with almost all of the body tissues. They carry oxygenated blood flow from the heart as well as carry back to the heart the unoxygenated blood for re-oxygenation.

About half of the volume of blood comprises red blood cells, white blood cells and platelets. Red blood cells, which contain a chemical substance called hemoglobin, pick up oxygen and carbon dioxide for transport. By changing shape, the white blood cells move about and can chase and engulf bacteria. Platelets help blood coagulate by forming fibrin, a network of fibers that trap blood cells and form a dam to hold back any further escape of blood from a scratch or wound.

The remaining portion of the blood is made up of watery fluid called plasma. In it are hundreds of different substances in suspension or solution, including blood-clotting proteins, antibodies, hormones, enzymes, minerals, and nutrients. Lymph is another fluid mixed with the plasma that carries these substances through the capillary walls and constantly bathes the cells of the body.

Blockages in the vascular system can cause serious and even life-threatening health problems. Blockage occurs when cholesterol-carrying fat globules (lipoproteins), plaque and fatty deposits attach to the inside walls of the vast vascular system within the human body. This build up can eventually prevent the free flow of oxygenated blood to the extremities, as well as the main arteries that feed the heart muscle. Plaque and cholesterol build up inside the walls of the human vascular system is the reason for atherosclerosis (hardening of the arteries) and high blood pressure, strokes and other cardiovascular conditions.

There remains a need for methods of treating and preventing heart disease, including atherosclerosis. There also remains a need for compositions and supplements that can be incorporated into a regular diet to reduce the likelihood of occurrence or ameliorate the symptoms of heart disease.

### BRIEF SUMMARY OF THE INVENTION

The present compositions can be used to limit and even help to reverse the effects of atherosclerosis, high blood pressure, strokes due to the build up of cholesterol, plaque and fatty deposits inside the vessels walls within the human body. The compositions and methods of using them can alleviate symptoms in a relatively short period of time, typically between about four to about twelve months with, at most, limited side effects. Even after symptoms may be alleviated, the compositions can be taken over an extended period of time to maintain good health and reduce the likelihood of the symptoms of heart disease occurring again.

The compositions include Generally Recognized as Safe (“GRAS”) ingredients that can to help reverse the effects of atherosclerosis in the human body. Without wishing to be bound by any theories, it is believed that the combination of the various amino acids, minerals, and essential fatty acids (saturated, monounsaturated, polyunsaturated) and carbohydrates of the nutritional compositions modify the viscosity of red blood cells and platelets in the blood. As a result, undesirable cholesterol, plaque and fatty deposits in the side walls of the vascular system are removed. The compositions can help to maintain healthy cholesterol levels by interacting with cholesterol and insulin to help reduce low density cholesterol and increase high density cholesterol. The compositions can help to lower triglyceride and homocysteine levels. The compositions and methods of using them may improve the elasticity of the vessels within the body, making them once again pliable and not ridged as with hardening of the arteries (atherosclerosis).

The nutritional compositions generally include proteins, phospholipids, vitamins and minerals and other essential nutrients for good health. The special blend of safe and natural ingredients is suitable for daily consumption over an extended period of time by a person seeking to avoid or treat the symptoms associated with heart disease.

Accordingly, it is an object to provide compositions and methods of using the compositions that will increase the longevity of and provide a better quality of life for a person by maintaining cardiovascular function and a healthy circulatory system.

It is another object to provide nutritional compositions and supplements that can be consumed as frequently as at least once a day to reduce the likelihood of the occurrence of and/or ameliorate the symptoms of heart disease.

It is another object to provide nutritional compositions and supplements that can be mixed with foods for consumption to reduce the likelihood of the occurrence of and/or ameliorate the symptoms of heart disease.

### DETAILED DESCRIPTION OF THE INVENTION

The nutritional compositions and supplements of the various embodiments of the invention are orally administered to treat or reduce the occurrence of heart disease. An unexpected discovery is that by combining specific natural ingredients, including soy-derived components, nutritional compositions can be prepared for reducing and treating the symptoms of heart disease such as atherosclerosis. The combination of ingredients simultaneously affects several different mechanisms that can result in atherosclerosis and associated diseases.

The nutritional compositions and supplements include protein, phospholipids and vitamins and minerals in amounts effective for reducing the likelihood of the occurrence of and/or ameliorating the symptoms of heart disease. In an important aspect, proteins and/or phospholipids derived from soy beans are used. The nutritional compositions can be taken directly or mixed with liquid or solid food products and consumed. They can be consumed once a day, such as in the morning, to mitigate the symptoms of atherosclerosis and other forms of heart disease.

As used herein, “to ameliorate the symptoms of heart disease” refers to a reduction, prevention, or elimination of one or more symptoms characteristic of heart disease. Such a reduction, prevention or elimination includes, but is not limited to a reduction in atherosclerotic plaque formation, reduction in hypertension, reduction in cholesterol and reduction in the occurrence of heart attack, angina and stroke and the like.

The terms “treat,” “treating,” “treatment,” and similar terms as used herein refer to the administration of the nutritional compositions to individuals, particularly humans, who are suffering from heart disease for alleviating, suppressing, inhibiting, or otherwise reducing the symptoms of heart disease, including atherosclerosis. The terms “treat,” “treating,” “treatment,” and similar terms also are used herein to refer to the prophylactic administration of the nutritional supplement to individuals who may be at risk of, or otherwise wish to avoid, heart disease, including atherosclerosis.

The nutritional compositions include a source of proteins and amino acids. Protein, for example, can help to raise the level of HDL cholesterol. Amino acids are the building blocks of protein and are an essential nutrient that is important to organ growth and maintenance. Preferably, the protein source and amino acids of the nutritional compositions are derived from soy beans. One suitable protein source is Supro® XT™ which is available from The Solae Company, St. Louis, Mo. In one aspect, the nutritional composition includes a source of proteins in the range of between about 21% to about 27% based on the weight of the composition, preferably between about 21.7% and about 26.6% based on the weight of the composition.

In one aspect, the protein source includes one or more amino acids, including but not limited to aspartic acid, threonine, serine, glutamic acid, praline, glycine, alanine, cystine, valine, methionine, isoleucine, leucine, tyrosine, phenylalanine, histidine, lysine, arginine and tryptophan. Aspartic acid is known for increasing energy and stamina. It combines with other amino acids to form molecules that absorb toxins and remove them from the blood stream. Threonine is known to help maintain the protein balance in the body. It is important in the formation of collagen and elastin, which is present in the walls of blood vessels, and thus to improving the elasticity of the vessels. Serine is known to help break down fats and fatty acids so that they are not stored as fat cells in the body. Glutamic acid is known to help metabolize sugars and fats. Proline is known for the formation of collagen and elastin. It helps to limit new build up of atherosclerotic deposits in the blood vessels and also helps to release lipoproteins that have deposited on the interior walls of the blood vessels. Glycine is known for increasing energy, limiting muscle degeneration and maintaining a healthy central nervous system and prostate. Alanine is known to assist with metabolism of glucose. Cystine is known for its detoxifying afftects, as well as for its impact on white blood cell activity. Valine is known for its cell stimulant effects. Methionine is known to assist with breakdown of fats to limit the build up of cholesterol and plaque in the liver and arteries, as well as to aid digestion. Isoleucine is known for hemoglobin formation. It also stabilizes and regulates blood sugar levels in the body. Leucine is known to assist with lowering blood sugar levels and healing of bones, wounds, skin and muscle tissue. Tyrosine is known for limiting the effects of anxiety, depression, allergies and headaches. Phenylalanine is known for treating depression, as well as for improving memory and reducing obesity. Histidine is known for tissue growth and repair. It also assists with treating allergies and producing red and white blood cells. Lysine is known for formation of collagen and assisting with stabilizing blood vessels. It helps to release lipoproteins that have deposited on the interior walls of the blood vessels. Arginine is known for assisting with formation of collagen and reduction of blood pressure. It aids in retardation of tumors and cancer, liver detoxification and maintenance of the immune system. Tryptophan is known for the production of niacin and enhancing the release of growth hormones necessary for the production of vitamin B₆ (pyridoxine).

The nutritional compositions include a source of phospholipids. Preferably, the phospholipids are derived from soy beans. Soy phospholipids may help reduce low density lipoproteins (LDLs) and increase high density lipoproteins (HDLs). Suitable phospholipids include, but are not limited to, phosphatidylcholine, phosphatidylethanolamine, phosphatidylinositol and phosphatideic acid. Phosphatidylcholine has the structure of a triglyceride in which a fatty acid moiety at the number one carbon of the glycerol is replaced by phosphocholine. Preferably, the compound has between about 13% to about 17% choline by weight. Choline is necessary in fat and cholesterol metabolism, assists in hormone production and minimizes excess fat in the liver. Phosphatidylethanolamine also is necessary in fat and cholesterol metabolism, assists in hormone production and minimizes excess fat in the liver. Phosphatidylinositol is known to assist in preventing hardening of the arteries and assists in fat and cholesterol metabolism. Phosphatideic acid assists in preventing hardening of the arteries and in synthesizing fatty acids. In one aspect, the nutritional composition includes phospholipids in the range of between about 48% to about 60% based on the weight of the composition, preferably between about 48.7% and about 59.6% based on the weight of the composition.

The nutritional compositions include a source of carnitine. One suitable source is L-carnitine L-tartrate. L-carnitine is a vitamin-like compound made from the amino acids lysine and methionine. It is found in animal-based sources of protein but not in plant-based sources. L-carnitine assists with burning fat globules for metabolic energy, so that the fat is not stored as body fat and/or in the vascular system as cholesterol and plaque build up. Carnitine helps to transport long-chain fatty acids and break down the fatty acid chains so that they can be utilized for energy. Carnitine enhances the removal of short- and medium-chain triglycerides from the mitochondria where fat metabolism occurs. Carnitine also helps to reduce the risk of heart disease and improve athletic ability. Carnitine, or L-carnitine, is a vitamin-like compound made in the body from the amino acids lysine and methionine. It is found in animal-based, not plant-based, sources of protein. It has been used to help with fat metabolism. In one aspect, the nutritional composition includes carnitine in the range of between about 3% to about 5% based on the weight of the composition, preferably between about 3.7% and about 4.6% based on the weight of the composition.

The concentration of carnitine can be an important factor governing the rate of fat metabolism. Fat metabolism occurs in the mitochondria via beta-oxidation. Fatty acids alone cannot penetrate the inner mitochondrial membrane, and a transport system that includes carnitine and at least three enzymes is required to effect passage. Carnitine receives an acyl group from coenzyme A (CoA) to commence transport and beta-oxidation. The reaction is mediated by the enzyme carnitine acyltransferase (CAT) I. The fat-carnitine complex (acylcarnitine) is transported across the inner mitochondrial membrane by the enzyme carnitine acylcarnitine translocase, followed by transfer of the fatty acid back to coenzyme A. The latter reaction is catalyzed by CAT II. The acyl-CoA that is formed is ultimately burned, yielding a large amount of metabolic energy in the form of adenosine triphosphate (ATP). More recently, the effect of carnitine on the removal of short- and medium-chain triglycerides has been shown. The reactions in which carnitine assists with removal of these triglycerides are catalyzed by a group of enzymes called short-chain and medium-chain carnitine acyltransferase. Transporting short-chain acyl units out of the mitochondria has the net effect of increasing the mitochondrial-free CoA levels, which stimulates fat metabolism.

The nutritional compositions include one or more vitamins and minerals or sources thereof including, but not limited to, vitamin A, vitamin C, thiamine (vitamin B₁), riboflavin (vitamin B₂), niacin (vitamin B₃), calcium d-pantothenate (vitamin B₅), pyridoxine (vitamin B₆), cyanocobalamin (vitamin B₁₂), vitamin E, folic acid, sodium, potassium, calcium, magnesium, phosphorus and iron. Vitamin A is known for tissue growth and development, as well as its function as an antioxidant. Vitamin C, or ascorbic acid, is known for tissue growth, formation of collagen, reduction of cholesterol levels and high blood pressure, protection against blood clotting, production of anti-stress hormones, and prevention of atherosclerosis. Thiamine is known for enhancing circulation and assisting in production of blood. Riboflavin is known for red blood cell formation and antibody production. Niacin is known for reduction of cholesterol and enhancing circulation. Vitamin E is known as an antioxidant for preventing cardiovascular disease, as well as for enhancing blood circulation and reducing blood pressure. Cyanocobalamin is known for preventing anemia and helping to metabolize carbohydrates. Folic acid is known for energy production, red blood cell formation and functioning as a coenzyme in DNA synthesis. Betaine is known to function closely with other nutrients including folic acid and vitamins B₆ and B₁₂ to help reduce levels of homocysteine, which is a naturally occurring amino acid that can be harmful to blood vessels.

Various minerals are important in the development of the nervous system and muscles, treatment of depression, maintaining a healthy immune system, preventing heart disease and reducing high blood pressure. Preferably, the minerals used in the nutritional compositions are derived from soy beans. Sodium is known for maintaining water balance and blood pH. Potassium is known to provide a regular heart rhythm and maintain stable blood pressure, prevent stroke, maintain water balance, assist with proper muscle contraction, regulate the transfer of nutrients into the cells, and for hormone secretion. Calcium is known for providing strong teeth and bones, as well maintaining a regular heartbeat, assisting with blood clotting and lowering blood pressure. It also provides energy and participates in protein restructuring of RNA and DNA, the activation of several enzymes including lipase, and transmission of nerve cell impulses. Magnesium is known to assist in preventing heart disease and high blood pressure, maintaining proper pH balance, protecting the arterial lining from stress from sudden blood pressure changes, promoting enzyme activity, and assisting with carbohydrate and mineral metabolism. Phosphorus is known for cell growth, conversion of food to energy and contraction of the heart muscle. Iron is known for assisting with production of hemoglobin and oxygenation of red blood cells and is essential for many enzymes and cell energy production.

One or more fatty acids also can be included in the nutritional compositions. Suitable fatty acids include, for example, linoleic 6 and linolenic 3, which also are referred to as omega fatty acids 6 and 3. These fatty acids are known to increase the activity of carnitine to enhance the burning of body fat.

Carbohydrates also are included in the nutritional compositions. Carbohydrates provide energy and take part in the metabolism of plant and animal matter including sugars, starches and cellulose. It has been shown that an increase in carbohydrate and fiber consumption coupled with reductions in dietary fat and cholesterol will reverse atherosclerosis and reduce the overall risk of death in many patients with advanced heart disease.

In other aspects, other suitable ingredients can be included in the nutritional compositions in accordance with techniques well known to persons skilled in the art. Other suitable ingredients can include flavorings, pharmaceutically acceptable carriers, additives to enhance the texture, stability and other physical characteristics of the compositions and/or the resulting food products after mixing, and additives to assist with mechanical processing of the nutritional compositions. Such ingredients can include, but are not limited to, sugars or other sweeteners, starches, cellulose, stabilizers, flavor compositions, colorants, and preservatives.

The nutritional compositions can be used as a food additive in a variety of foodstuffs and beverages. They can be used with dry foods such as cereal or mixed with the ingredients for baked goods including breads and cookies. They can be mixed with liquids including water and fruit juices to make a beverage. They can be formed into tablets, capsules, chewable wafers or other forms that can be directly orally administered.

### EXAMPLES

The following examples further illustrate embodiments of the present invention but are not be construed as in any way limiting the scope of the present invention as set forth in the appended claims.

### Example 1

This example illustrates a first composition for reducing the likelihood of the occurrence of and/or ameliorating the symptoms of heart disease. The composition includes the formulation set forth in Table 1.

TABLE 1

| | Per 100 grams | Low | High |
|---|---|---|---|
| Calories (kcal) | 558.2 | 502.4 | 614.0 |
| Calories from fat (kcal) | 307.2 | 276.5 | 337.9 |
| Total Fat (g) | 33.9 | 30.5 | 37.3 |
| Saturated (g) | 8.3 | 7.5 | 9.2 |
| Stearic(g) | 1.3 | 1.2 | 1.4 |
| Palmitic (g) | 7.0 | 6.3 | 7.7 |
| Monounsaturated (g) | 3.2 | 2.9 | 3.5 |
| Oleic (g) | 3.2 | 2.9 | 3.5 |
| Polyunsaturated (g) | 22.4 | 20.2 | 24.6 |
| Linoleic [ω-6] (g) | 18.6 | 16.7 | 20.4 |
| Linoleic [ω-3] (g) | 3.8 | 3.5 | 4.2 |
| Total Carbohydrates (g) | 5.1 | 4.6 | 5.6 |
| Dietary Fiber (g) | * | * | * |
| Sugars (g) | 2.6 | 2.3 | 2.8 |
| Vitamin A (mg) | * | * | * |
| Vitamin C (mg) | * | * | * |
| Moisture (g) | <1 | * | * |
| Thiamine (μg) | * | * | * |
| Riboflavin (μg) | * | * | * |
| Niacin (μg) | * | * | * |
| Folic Acid (μg) | * | * | * |
| Vitamin E (IU) | 3.8 | 3.5 | 4.2 |
| Vitamin B 12 (μg) | * | * | * |
| Phosphatidylcholine (%) | 15% wt | 13.5% wt | 16.5% wt |
| Phosphatidylethanolamine (%) | 13% wt | 12% wt | 14% wt |
| Phosphatidylinositol (%) | 9% wt | 8% wt | 10% wt |
| Phosphatideic Acid (%) | 5% wt | 5% wt | 5% wt |
| Sodium (mg) | 384.04 | 346.6 | 422.4 |
| Calcium (mg) | 125.9 | 113.3 | 138.4 |
| Iron (mg) | 5.2 | 4.6 | 5.7 |
| Potassium (mg) | 1154.5 | 1039.1 | 1270.0 |
| Phosphorus (mg) | 2181.0 | 1962.9 | 2399.1 |
| Magnesium (mg) | 18.1 | 16.3 | 19.9 |
| Aspartic Acid (g) | 3.5 | 3.1 | 3.8 |
| Threonine (g) | 1.1 | 1.0 | 1.2 |
| Serine (g) | 1.6 | 1.4 | 1.7 |
| Glutamic Acid (g) | 5.8 | 5.2 | 6.3 |
| Proline (g) | 1.6 | 1.4 | 1.8 |
| Glycine (g) | 1.2 | 1.1 | 1.4 |
| Alanine (g) | 1.2 | 1.1 | 1.4 |
| Cystine (g) | 0.3 | 0.3 | 0.3 |
| Valine (g) | 1.4 | 1.3 | 1.5 |
| Methionine (g) | 0.4 | 0.4 | 0.4 |
| Isoleucine (g) | 1.4 | 1.3 | 1.5 |
| Leucine (g) | 2.4 | 2.2 | 2.6 |
| Tyrosine (g) | 1.2 | 1.1 | 1.3 |
| Phenylalanine (g) | 1.6 | 1.4 | 1.7 |
| Histidine (g) | 0.8 | 0.7 | 0.9 |
| Lysine (g) | 1.9 | 1.7 | 2.0 |
| Arginine (g) | 2.3 | 2.1 | 2.6 |
| Tryptophan (g) | 0.3 | 0.3 | 0.3 |
| Isoflavone (g) | 0.6 | 0.5 | 0.6 |
| L-Carnitine L-Tartrate (g) | 4.0 | 3.6 | 4.4 |
| Natural Flavorings (g) | 2.5 | 2.3 | 2.8 |
| Sweetener (g) | 0.5 | 0.5 | 0.5 |

The above ingredients are mixed into a dry composition. Approximately 15 grams of the composition is mixed with a liquid, including water or juice, to form a beverage that is consumed once a day.

### Example 2

This example illustrates a second composition for reducing the likelihood of the occurrence of and/or ameliorating the symptoms of heart disease. The composition includes the formulation set forth in Table 2.

TABLE 2

| Ingredient | % (weight) | Weight (g) | Low (g) | High (g) |
|---|---|---|---|---|
| L-Carnitine Tartrate | 4.17 | 18.33 | 16.50 | 20.16 |
| Phospholipids | 54.17 | 238.33 | 214.50 | 262.16 |
| Soy Protein | 24.17 | 106.33 | 95.70 | 116.96 |
| Fructose | 13.46 | 59.24 | 53.32 | 65.16 |
| Sweetener | 0.23 | 1.03 | 0.93 | 1.13 |
| Flavor | 2.65 | 11.66 | 10.49 | 12.83 |
| Thiamin HCl | 0.02 | 0.09 | 0.08 | 0.10 |
| Riboflavin | 0.02 | 0.08 | 0.07 | 0.09 |
| Inositol Nicontinate | 0.73 | 3.19 | 2.87 | 3.51 |
| Calcium d-Pantothenate | 0.07 | 0.29 | 0.26 | 0.32 |
| Pyridoxine HCl | 0.02 | 0.10 | 0.09 | 0.11 |
| Folic Acid | <0.01 | 0.01 | 0.01 | 0.01 |
| Cyanocobalamin 1% | <0.01 | <0.01 | <0.01 | <0.01 |
| Betaine HCl | 0.30 | 1.31 | 1.18 | 1.44 |

The above ingredients are mixed into a dry composition. Approximately 15 grams of the composition is mixed with a liquid, including water or juice, to form a beverage that is consumed once a day.

Many modifications and variations may be made in the techniques and compositions described and illustrated herein without departing from the spirit and scope of the present invention. Accordingly, the techniques and compositions described and illustrated herein should be understood to be illustrative only and not limiting upon the scope of the present invention.

1. A nutritional composition for treating atherosclerosis comprising:
a protein source;
phospholipids;
a source of carnitine; and
a vitamin composition.

2. The nutritional composition according to claim 1 further including a source of betaine.

3. The nutritional composition according to claim 1 wherein the protein source and phospholipids are derived from soy beans.

4. The nutritional composition according to claim 1 wherein the protein source includes one or more amino acids selected from the group consisting of aspartic acid, threonine, serine, glutamic acid, praline, glycine, alanine, cystine, valine, methionine, isoleucine, leucine, tyrosine, phenylalanine, histidine, lysine, arginine and tryptophan and combinations thereof.

5. The nutritional composition according to claim 1 wherein the phospholipids are selected from the group consisting of phosphatidylcholine, phosphatidylethanolamine, phosphatidylinositol and phosphatideic acid and combinations thereof.

6. The nutritional composition according to claim 1 wherein the vitamin composition includes one or more vitamins selected from the group consisting of vitamin A, vitamin C, thiamine (vitamin B₁), riboflavin (vitamin B₂), niacin (vitamin B₃), calcium d-pantothenate (vitamin B₅), pyridoxine (vitamin B₆), cyanocobalamin (vitamin B₁₂), vitamin E, folic acid and vitamin E and combinations thereof.

7. The nutritional composition according to claim 1 further including a mineral composition having one or more minerals selected from the group consisting of sodium, potassium, calcium, magnesium, phosphorus and iron and combinations thereof.

8. A nutritional composition for treating heart disease comprising:
a source of soy protein;
soy-derived phospholipids;
one or more vitamins selected from the group consisting of vitamin A, vitamin C, thiamine (vitamin B₁), riboflavin (vitamin B₂), niacin (vitamin B₃), calcium d-pantothenate (vitamin B₅), pyridoxine (vitamin B₆), cyanocobalamin (vitamin B₁₂), vitamin E, folic acid and vitamin E and combinations thereof; and
a source of carnitine,
each of the components of the nutritional composition present in an amount effective for reducing and ameliorating the symptoms of atherosclerosis.

9. The nutritional composition according to claim 8 wherein the source of soy protein is present in an amount of between about 21% to about 27% based on the weight of the composition.

10. The nutritional composition according to claim 8 wherein the soy-derived phospholipids are present in an amount of between about 48% to about 60% based on the weight of the composition.

11. The nutritional composition according to claim 8 wherein the source of carnitine is present in an amount of between about 3% to about 5% based on the weight of the composition.

12. The nutritional composition according to claim 8 further including a source of betaine.

13. The nutritional composition according to claim 8 wherein the soy-derived phospholipids are selected from the group consisting of phosphatidylcholine, phosphatidylethanolamine, phosphatidylinositol and phosphatideic acid and combinations thereof.

14. The nutritional composition according to claim 8 wherein the source of soy protein includes one or more amino acids selected from the group consisting of aspartic acid, threonine, serine, glutamic acid, praline, glycine, alanine, cystine, valine, methionine, isoleucine, leucine, tyrosine, phenylalanine, histidine, lysine, arginine and tryptophan and combinations thereof.

15. The nutritional composition according to claim 8 further including a mineral composition having one or more minerals selected from the group consisting of sodium, potassium, calcium, magnesium, phosphorus and iron and combinations thereof.

16. A method of reducing or ameliorating the symptoms of heart disease comprising administering an effective amount of a composition of soy-based protein, soy-based phospholipids, a source of carnitine, and one or more vitamins selected from the group consisting of vitamin A, vitamin C, thiamine (vitamin B₁), riboflavin (vitamin B₂), niacin (vitamin B₃), calcium d-pantothenate (vitamin B₅), pyridoxine (vitamin B₆), cyanocobalamin (vitamin B₁₂), vitamin E, folic acid and vitamin E.

17. The method according to claim 16 wherein the soy-based protein is administered in an amount of between about 21% to about 27% based on the weight of the composition.

18. The method according to claim 16 wherein the soy-based phospholipids are administered in an amount of between about 48% to about 60% based on the weight of the composition.

19. The nutritional composition according to claim 16 wherein the source of carnitine is administered in an amount of between about 3% to about 5% based on the weight of the composition.

20. The method according to claim 16 wherein the composition further includes a source of betaine.
